(19) United States
(12) Patent Application Publication
(10) Pub. No.: US 2003/0017177 A1
(43) Pub. Date: Jan. 23, 2003

(54) Reduction of skin pore size using alkanolamines

(76) Inventor: Nicholas V. Perricone, Guilford, CT (US)

Correspondence Address: MARY M. KRINSKY, Ph. D., J.D., PATENT ATTORNEY, 79 TRUMBULL STREET, NEW HAVEN, CT, 06511, US

(21) Appl. No.: 09/900,680

(22) Filed: Jul. 6, 2001

(51) Int. Cl. A61K 31/13; A61K 7/00; A61K 31/198

(52) U.S. Cl. 424/401; 514/667; 514/561

(57) ABSTRACT

Skin pore size, particularly on the face and scalp, is reduced by the topical application of compositions containing an alkanolamine such as ethylaminoethanol, methylaminoethanol, dimethylaminoethanol, isopropanolamine, triethanolamine, isopropanoldimethylamine, ethylethanolamine, 2-butanolamine, choline, serine, and mixtures thereof. Dimethylaminoethanol in amounts ranging from about 0.1% to about 10% by weight of the total composition is particularly preferred. Adjunct ingredients such as tyrosine, ascorbyl palmitate, and glycolic acid may be added to pore-reducing formulations.

### BACKGROUND OF THE INVENTION

1. Field of the Invention

This invention relates to compositions and methods for the reduction of skin pore size and the improvement of overall skin appearance, particularly on the face.

2. Description of Related Art

Manunalian kin is characterized by thousands of pores which function as openings for sebaceous (oil-producing) and eccrlne (sweat-producing) glands and hair follicles. Pores generally serve as openings for the secretion of glandular products such as sebum, and orifices for externally applied substances, including lotions, creams, and cosmetics. Skin pores are large and numerous on the face and scalp, areas of maximum exposure. For facial areas, the density ranges from 400 to 800 pores/cm², compared with about 50 pores/cm² on the arms and legs. The forehead, nose, and nasolabial folds are the areas of highest pore concentration.

Pores have a defined size which is susceptible to measurement. Pore size is largely determined by genetic, environmental, and physiological factors. Visible pore diameter is often proportional to the size of subcutaneous sebaceous glands, and increased pore size is frequently associated with hyperactive sebaceous glands, including increased glandular activity and higher sebum production that occurs in adolescence, and with debris accumulation such as that observed in aging, when sebum production slows sufficiently to inhibit the constant stratum corneum shedding of normal youthful skin. Hyperactive sebaceous glands generate larger amounts of sebum which expands the pilary canal and dilates pore diameter to accommodate greater internal pressure. The aging process causes deterioration of the dermal elements surrounding the follicle. These changes are manifested by internal collapse of supporting skin structure and expansion of the follular canal, resulting in pore dilation and greater visibility on the skin surface. The visual appearance of skin pores also partially depends upon the texture of surrounding surfaces. Rough skin scatters light in a manner which emphasizes openings on the skin surface, so pores appear larger.

Current treatments for enlarged pores are directed primarily to cleaning the skin to facilitate sebum and debris removal. Frequent washing is recommended for persons with oily skin, and washing with skin cleaners containing hydrating agents, for persons with normal and dry skin. Sebum production is commonly curbed using drying agents such as alcohol and benzoyl peroxide. Special formulations containing amphoteric, cationic, and anionic surfactants (and optional active ingredients) that don't overdry or irritate skin have been suggested by McAtee, et al., for reducing pore size, both as leave-on products or products that are rinsed or wiped from the skin after use (U.S. Pat. No. 5,607,980; this and other patents cited below are expressly incorporated herein in their entireties by reference). Non-vasodilating vitamin B₃ compounds, particularly niacinamide, alone, but preferably in combination with retinoids, have been suggested for reducing skin pore size (U.S. Pat. No. 6,217,888 to Oblong, et al.). A composition comprising an oil absorbing powder, a botanical astringent, and a biological compound that alters the structure of the skin and/or the function of the sebaceous glands, such as salicylic acid, farnesyl acetate, panthetine triacetate, pyridoxine hydrochloride (vitamin B₆), biotin, lysine carboxy-methyl cysteinate, and mixtures thereof, has also been suggested for visibly reducing the size of skin pores (U.S. Pat. Nos. 5,415,861 and 5,472,699 to Duffy, et al.).

It would be desirable to have alternative treatments for reducing skin pore size and improving overall skin appearance, particularly compositions that physically contract pores rather than just clean them.

### BRIEF SUMMARY OF THE INVENTION

It is a primary objective of the invention to provide a pore-reducing composition and methods for its use.

These and other objectives are accomplished by this invention, which provides compositions containing an alkanolamine such as ethylaminoethanol, methylaminoethanol, dimethylaminoethanol, isopropanolamine, triethanolamine, isopropanoldimethylamine, ethylethanolamine, 2-butanolamine, choline, serine, and mixtures thereof, which are used on mammalian skin for the visible reduction of pore size. Dimethylaminoethanol is particularly preferred. Amounts of active alkanolamine ingredient range from about 0.1 to about 10%, more narrowly from about 1% to about 3%, by weight of the total composition. Adjunct ingredients such as tyrosine, a fatty acid ester of ascorbic acid, e.g., ascorbyl palmitate, and/or an α-hydroxy acid, e.g., glycolic acid may be added to pore-reducing formulations of the invention. Methods and compositions of the invention are particularly efficacious for reducing pores on the face and scalp, including the enlarged pores often observed in adolescence and aging.

### BRIEF DESCRIPTION OF THE INVENTION

Methods of the invention involve the topical administration of dimethylaminoethanol and/or other structurally related alkanolamines, or their biologically equivalent derivatives, for the visible reduction of skin pores and the overall improvement in skin appearance.

In the practice of the invention, compositions containing an effective amount of an alkanolamine of the formula

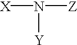

wherein X, Y and Z are selected from the group consisting of hydrogen, C₁-C ₃ alkyl groups, C₂-C₄ alkanol group, wherein at least one of X, Y, or Z is a C₂-C₄ alkanol group bearing at least one hydroxyl group and optionally at least one carboxyl group, are applied to mammalian skin to visibly reduce pore size. Useful compounds for the invention include, but are not limited to, ethylaminoethanol, methylaminoethanol, dimethylaminoethanol, isopropanolamine, triethanolamine, isopropanoldimethylamine, ethylethanolamine, 2-butanolamine, choline, serine, and mixtures thereof. Many preferred embodiments employ methylaminoaminoethanol, dimethylaminoethanol, ethylaminoethanol, and/or triethanolamine; particularly preferred is dimethylaminoethanol (DMAE).

However, only effective amounts of alkanolamines are needed to shrink pores, so generally topical application is accomplished in association with a carrier, and particularly one in which the alkanolamine active ingredient is soluble per se or is effectively solubilized (e.g., as an emulsion or microemulsion). Where employed, the carrier is inert in the sense of not bringing about a deactivation or oxidation of the polyphenol, and in the sense of not bringing about any adverse effect on the skin areas to which it is applied. In one preferred practice of the invention, dimethylaminoethanol is applied in admixture with a dermatologically acceptable carrier or vehicle (e.g., as a lotion, cream, ointment, soap, stick, or the like) so as to facilitate topical application and, in some cases, provide additional therapeutic effects as might be brought about, e.g., by moisturizing of the affected skin or scalp areas. While the alkanolamine carrier for dermatological compositions can consist of a relatively simple solvent or dispersant such as water, it is generally preferred that the carrier comprise a composition more conducive to topical application, and particularly one which will form a film or layer on the skin to which it is applied so as to localize the application and provide some resistance to washing off by immersion in water or by perspiration and/or aid in the percutaneous delivery of the active agent. Many preparations are known in the art, and include lotions containing oils and/or alcohols and emollients vegetable oils, hydrocarbon oils and waxes, silicone oils, animal or marine fats or oils, glyceride derivatives, fatty acids or fatty acid esters or alcohols or alcohol ethers, lecithin, lanolin and derivatives, polyhydric alcohols or esters, wax esters, sterols, phospholipids and the like, and generally also emulsifiers (nonionic, cationic or anionic), although some of the emollients inherently possess emulsifying properties. These same general ingredients can be formulated into a cream rather than a lotion, or into gels, or into solid sticks by utilization of different proportions of the ingredients and/or by inclusion of thickening agents such as gums or other forms of hydrophilic colloids. One preferred embodiment is an oil-in-water cream. Such compositions are referred to herein as dermally or dermatologically acceptable carriers, and are formulated using conventional techniques known to those of ordinary skill in the art.

Suitable carriers include water, alcohols, oils and the like, chosen for their ability to dissolve or disperse polyphenol and any other ingredients used in the treatment. Generally, even low concentrations of active ingredient in a carrier are suitable, depending upon the application regimen and adjunct ingredients employed. Many embodiments contain from about 0.1% to about 10% by weight, more narrowly from about 0.25% to about 5% to 7% by weight, and in many cases from about 1% to about 3% by weight, alkanolamine such as dimethylaminoethanol in the total composition. Chronic conditions such as enlarged pores observed on the faces of persons with oily skin typically require a lower concentration of active alkanolamine ingredient than to acute conditions such as enlarged pores observed in adolescence. As a practical matter, however, to avoid the need for repeated application, it is desirable that the topically applied composition (i.e., alkanolamine plus carrier) be formulated to contain at least about 1% by weight alkanolamine, and many embodiments contain more than 1 weight % alkanolamine. One efficacious embodiment contains from about 2% to about 5% by weight alkanolamine.

Generally in the practice of methods of the invention, the composition is topically applied to the affected skin areas in a predetermined or as-needed regimen either at intervals by application of a lotion or the like, it generally being the case that gradual improvement is noted with each successive application. Insofar as has been determined based upon clinical studies to date, no adverse side effects are encountered.

Some embodiments of this invention contain at least one other adjunct ingredient in addition to alkanolamine. Adjunct ingredients include, but are not limited to, α-hydroxy acids, tyrosine, and fatty acid esters of ascorbic acid. Many embodiments employ more than one adjunct ingredient. Where employed, adjunct ingredients are anticipated to have additive effects if not synergistic effects due to different mechanisms of action.

As used herein, the term “α-hydroxy acid” has reference to and encompasses the general class of organic compounds containing at least one hydroxy group and at least one carboxyl group, and wherein at least one hydroxyl group is located on the α-carbon atom. Typically, the compounds are organic acids having at least one carboxylic acid group and at least one hydroxyl group on the α-carbon atom, and may contain other functional groups including additional hydroxyl and carboxylic acid moieties. Preferred α-hydroxy acids and/or α-hydroxy acid derivatives are less bulky structurally so that they penetrate the skin well, and thus have a backbone of from one to three carbon atoms such as those set out in U.S. Pat. No. 5,965,618 at column 6 lines 4 to 29. Where employed, glycolic and/or lactic acid or their derivatives are preferred; glycolic acid is especially efficacious. Glycolic acid or other α-hydroxy acids are typically present in amounts ranging from about 1% to about 10%, more narrowly from about 3% to about 7% of the total composition.

Tyrosine may be present in pore-reducing compositions of the invention in amounts typically from about 0.01% to about 6%, more narrowly from about 0.03% to about 5% by weight, and, in some embodiments, about 0.5% by weight, based on the total composition.

Fat-soluble fatty acid esters of ascorbic acid (vitamin C) is employed as an adjunct ingredient in other embodiments, alone or in combination with α-hydroxy acids. The more oxidation-resistant saturated fatty acid esters of ascorbic acid are preferred, including, but not limited to, ascorbyl laurate, ascorbyl myristate, ascorbyl palmitate, ascorbyl stearate, and ascorbyl behenate. Ascorbyl palmitate is used in one embodiment. As denoted herein, where fatty acid esters are described, e.g., ascorbyl stearate, compositions having predominantly that ester, e.g., predominantly stearate, are included. The esters may be prepared using hydrogenated oils or fats, or fractions thereof, and contain small amounts of another ester. Ascorbyl stearate prepared using canola, for example, commonly contain about 4% ascorbyl palmitate. It is an advantage of the invention that where fatty acid esters of ascorbic acid are employed as an adjunct ingredient, they help stabilize the alkanolamine in the composition. Ascorbyl palmitate and the like ascorbyl esters are typically present in amounts ranging from about 0.5% to about 15%, preferably from about 1% to about 7% to 10%, of the total composition.

Pore-reducing topical compositions of the invention can comprise additional ingredients commonly found in skin care compositions, such as, for example, emollients, skin conditioning agents, emulsifying agents, humectants, preservatives, antioxidants, perfumes, chelating agents, etc., provided that they are physically and chemically compatible with other components of the composition. Preservatives include, but are not limited to, C₁-C₃ alkyl parabens and phenoxyenthanol, typically present in an amount ranging from about 0.5% to about 2.0% by weight percent, based on the total composition. Emollients, typically present in amounts ranging from about 0.01% to 5% of the total composition include, but are not limited to, fatty esters, fatty alcohols, mineral oils, polyether siloxane copolymers, and mixtures thereof. Humectants, typically present in amounts ranging from about 0.1% to about 5% by weight of the total composition include, but are not limited to, polyhydric alcohols such as glycerol, polyalkylene glycols (e.g., butylene glycol, propylene glycol, dipropylene glycol, polypropylene glycol, and polyethylene glycol) and derivatives thereof, alkylene polyols and their derivatives, sorbitol, hydroxy sorbitol, hexylene glycol, 1,3-dibutylene glycol, 1,2,6-hexanetriol, ethoxylated glycerol, propoxylated glycerol, and mixtures thereof. Emulsifiers, typically present in amounts from about 1% to about 10% by weight of the composition, include, but are not limited to, stearic acid, cetyl alcohol, stearyl alcohol, steareth 2, steareth 20, acrylates/C₁₀₋₃₀ alkyl acrylate crosspolymers, and mixtures thereof. Chelating agents, typically present in amounts ranging from about 0.01% to about 2% by weight, include, but are not limited to, ethylenediamine tetraacetic acid (EDTA) and derivatives and salts thereof, dihydroxyethyl glycine, tartaric acid, and mixtures thereof. Antioxidants, typically present in an amount ranging from about 0.02% to about 0.5% by weight of the composition, include, but are not limited to, butylated hydroxy toluene (BHT); vitamin C and/or vitamin C derivatives, such as fatty acid esters of ascorbic acid, particularly asocorbyl palmitate; butylated hydroanisole (BHA); phenyl-α-naphthylamine; hydroquinone; propyl gallate; nordihydroquiaretic acid; vitamin E and/or derivatives of vitamin E, including tocotrienol and/or tocotrienol derivatives; calcium pantothenates; green tea extracts; mixed polyphenols; and mixtures of any of these. As mentioned above, particularly preferred antioxidants are those that provide additional benefits to the skin such as ascorbyl palmitate and tocotrienol, and formulations using tocotrienol-enriched oils. (See additional ingredients and methods in U.S. Pat. Nos. 4,775,530, 5,376,361, 5,409,693, 5,545,398, 5,574,063, 5,643,586, 5,709,868, 5,879,690, 5,965,618, 5,968,618, 6,051,244, 6,162,419, and 6,191,121 to Perricone).

Buffering agents are employed in many compositions. Preferably, the amount of buffering agent is one that results in compositions having a pH ranging from about 4.5 to about 8.5, more preferably from about 5.5 to about 8.5, most preferably from about 6.5 to about 8.0. Typical buffering agents are chemically and physically stable agents commonly found in cosmetics, and can include compounds that are also adjunct ingredients such as citric acid, malic acid, and glycolic acid buffers.

Topical application of a composition of the invention containing about 2% by weight DMAE typically results in a visible contraction of skin pores within about 10 to 15 minutes after application, resulting in a smoother appearing complexion. The results are cumulative. With continued applications, pores become smaller and tighter over time. It is an advantage of the invention that it reduces pore size, a significant cosmetic concern to many people, particularly pores on the face. It is a further advantage of the invention that this effect coincides with other beneficial results obtained by applying alkanolamines to skin, including the treatment of skin damage and aging (U.S. Pat. No. 5,554,647 to Perricone) and scar treatment (co-pending U.S. application Ser. No. 09/875,317 to Perricone).

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

1. A method for visibly reducing pore size on mammalian skin comprising applying to skin a composition containing an effective amount of an alkanolamine of the formula 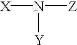
wherein X, Y and Z are selected from the group consisting of hydrogen, C₁-C₃ alkyl groups, C₂-C₄ alkanol group, wherein at least one of X, Y, or Z is a C₂-C₄ alkanol group bearing at least one hydroxyl group and optionally at least one carboxyl group.

2. A method according to claim 1 wherein the alkanolamine is selected from the group consisting of ethylaminoethanol, methylaminoethanol, dimethylaminoethanol, isopropanolamine, triethanolamine, isopropanoldimethylamine, ethylethanolamine, 2-butanolamine, choline, serine, and mixtures thereof.

3. A method according to claim 2 wherein the alkanolamine is dimethylaminoethanol.

4. A method according to claim 1 wherein the alkanolamine is present in the composition in an amount ranging from about 0.1% to about 10% by weight of the composition.

5. A method according to claim 4 wherein the alkanolamine is present in the composition in an amount ranging from about 1% to about 3% by weight of the composition.

6. A method according to claim 1 wherein the composition further comprises at least one adjunct ingredient selected from the group consisting of tyrosine, an α-hydroxy acid, a fatty acid ester of ascorbic acid, and mixtures of any of these.

7. A method according to claim 6 wherein the composition comprises tyrosine.

8. A method according to claim 6 wherein the composition comprises an α-hydroxy acid.

9. A method according to claim 8 wherein the α-hydroxy acid is glycolic acid.

10. A method according to claim 6 wherein the composition comprises a fatty acid ester of ascorbic acid.

11. A method according to claim 10 wherein the fatty acid ester of ascorbic acid is ascorbyl palmitate.

12. A method according to claim 1 wherein the composition further comprises another ingredient selected from the group consisting of a preservative, an emollient, an antioxidant, an emulsifier, a humectant, a buffer, and mixtures of any of these.

13. A method according to claim 1 for reducing pores on the face and scalp.

14. A method for visibly reducing pores on mammalian skin comprising applying to the skin a composition containing from about 0.1% to about 10% by weight of an alkanolamine selected from the group consisting of ethylaminoethanol, methylaminoethanol, dimethylaminoethanol, isopropanolamine, triethanolamine, isopropanoldimethylamine, ethylethanolamine, 2-butanolamine, choline, serine, and mixtures thereof.

15. A method according to claim 14 wherein the composition comprises dimethylaminoethanol.

16. A method according to claim 14 wherein the composition comprises from about 1% to about 3% by weigt alkanolamine.

17. A method according to claim 14 wherein the composition further comprises at least one adjunct ingredient selected from the group consisting of tyrosine, glycolic acid, ascorbyl palmitate, and mixtures of any of these.

18. A method according to claim 14 which reduces facial pores.

19. A method for visibly reducing facial or scalp pores on mammalian skin comprising applying to the skin a composition containing from about 0.10% to about 10% by weight dimethylaminoethanol and at least one adjunct ingredient selected from the group consisting of from about 0.01% to about 5% by weight tyrosine, from about 1% % to about 10% by weight of glycolic acid, from about 0.5 to about 15% by weight ascorbyl palmitate and mixtures of any of these.

20. A method according to claim 19 wherein the composition contains from about 1% to about 3% dimethylaminoethanol and at least one adjunct ingredient selected from the group consisting of from about 0.04% to about 3% by weight tyrosine, from about 3% to about 7% by weight of glycolic acid, from about 1 to about 7% by weight ascorbyl palmitate and mixtures of any of these.
